(19) United States
(12) Patent Application Publication
(10) Pub. No.: US 2007/0142819 A1
(43) Pub. Date: Jun. 21, 2007

(54) Bifurcated catheter for agent delivery and method of agent delivery

(76) Inventors: Fozan O. El-Nounou, Santa Clara, CA (US); Florian Niklas Ludwig, Mountain View, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/314,059

(22) Filed: Dec. 20, 2005

(51) Int. Cl. A61M 31/00

(52) U.S. Cl. 604/509; 604/101.04

(57) ABSTRACT

A bifurcated catheter having a branched distal shaft section and one or more porous or nonporous balloons, and combinations thereof, which is configured for delivery of an agent to a patient's bifurcated body lumen. Another aspect of the invention is directed to a method of delivering an agent to a patient's body lumen which facilitates maximizing the efficiency of drug uptake into the tissue at the desired site within the patient's body lumen.

### FIELD

The present invention relates generally to medical devices, and more particularly to a catheter for delivery of an agent to the coronary or peripheral vasculature.

### BACKGROUND OF THE INVENTION

In the treatment of diseased vasculature, therapeutic agents have commonly been administered, typically as part of other interventional therapies such as angioplasty or stent delivery. Local, as opposed to systemic delivery is a preferred method of treatment in that smaller total levels of medication are administered in comparison to systemic dosages, yet are concentrated at a specific site. As a result, local delivery produces fewer side effects and achieves more effective results.

A variety of methods and devices have been proposed for percutaneous drug delivery to a diseased region of the vasculature. For example, catheters having porous balloons can be used to deliver a therapeutic agent infused into the inflatable interior of the porous balloon and through the porous wall of the balloon. Alternatively, prostheses such as stents or other implantable devices provide for local drug delivery when coated or otherwise made to include a therapeutic agent which elutes from the implanted prosthesis. Another suggested method involves the use of one or more catheters having multiple balloons. The diseased region is isolated by inflating the balloons on either side of the diseased region, and the therapeutic agent is infused through a lumen of the catheter shaft and into the isolated diseased region from a delivery port on the catheter shaft located between the balloons.

One difficulty has been maximizing the amount of drug taken-up and retained at the diseased site, while minimizing the wash out of large amounts of drug downstream of the treatment site. Drug wash out reduces the efficiency of local intravascular drug delivery, in addition to causing potentially harmful systemic exposure to the drug. Therefore, it would be a significant advance to provide an improved device and method for providing therapy to a desired location within a patient's body lumen.

### SUMMARY OF THE INVENTION

The invention is directed to a bifurcated catheter having a branched distal shaft section and one or more porous or nonporous balloons, and combinations thereof, which is configured for delivery of an agent to a patient's bifurcated body lumen. Another aspect of the invention is directed to a method of delivering an agent to a patient's body lumen which facilitates maximizing the efficiency of drug uptake into the tissue at the desired site within the patient's body lumen.

In a first embodiment, a multi-balloon bifurcated catheter of the invention generally comprises an elongated shaft having an inflation lumen, a guidewire lumen, a proximal shaft section with a proximal section of the inflation lumen therein, a branched distal shaft section having a first branch with a first distal section of the inflation lumen and a second branch with a second distal section of the inflation lumen, the first and second distal sections of the inflation lumen being in fluid communication with the proximal section of the inflation lumen, and having an agent delivery lumen extending in the proximal shaft section to one or more distal ports which is/are preferably located adjacent to a proximal end(s) of the branched distal shaft section. A proximal balloon is on the proximal shaft section located proximal to the distal port of the agent delivery lumen, a first distal balloon is on the first branch, and a second distal balloon is on the second branch. The proximal balloon and first and second distal balloons each have an interior in fluid communication with the shaft inflation lumen. In one embodiment, the balloons are in fluid communication with the same inflation lumen and are therefore configured for simultaneous inflation. In alternative embodiments, the shaft has multiple (two or more), separate inflation lumens allowing the balloons to be inflated independently of one another.

The first and second distal balloons are solid-walled occlusion balloons with fluid tight interiors configured to inflate into contact with the body lumen wall, so that agent introduced into the body lumen via the agent delivery lumen of the catheter is prevented from flowing across the inflated first and second distal balloons. In a presently preferred embodiment, the proximal balloon is also a solid-walled occlusion balloon. With the proximal occlusion balloon and the first and second distal occlusion balloons inflated at a bifurcation of the patient's body lumen, agent delivered out the distal port of the agent delivery lumen into the bifurcated body lumen is confined within both the main and side branch of the bifurcated body lumen, between the inflated balloons. The first and second distal balloons are sealingly secured to the separate branches of the distal shaft, at locations spaced a sufficient distance distally apart from the proximal balloon such that the first and second distal balloons can be inflated without contacting oneanother and without contacting the bifurcation apex or crux (i.e., at the edge of the ostium defining the opening into the side branch vessel from the main branch vessel of the body lumen). Thus, in a method of using the multi-balloon bifurcated catheter, the first distal balloon is inflated in a side branch of the bifurcation and the second distal balloon is inflated in a main branch of the bifurcation with the inflated balloons typically longitudinally displaced from the crux of the bifurcation of the body lumen, so that diseased tissue at the crux of the bifurcation is exposed to a therapeutic agent contained in both the main and side branches of the body lumen, to thereby treat the diseased tissue. In contrast, methods of dilating a lesion or implanting a prosthesis at a bifurcation as previously described, such as a “kissing balloon” technique, are typically configured to inflate one or more balloons against the crux of the bifurcation.

In a presently preferred embodiment, the multi-balloon bifurcated catheter is a rapid-exchange catheter having a guidewire lumen which extends from a distal port at a distal end of the first branch of the catheter to a proximal port in the proximal shaft section. The rapid exchange guidewire lumen extends from the proximal port located proximal to the proximal balloon to the distal port located distal to one of the distal balloons. As a result, agent delivered to the treatment region isolated between the proximal balloon and the first and second distal balloons is prevented from washing out of the desired region through the rapid exchange guidewire lumen. In contrast, wash out of the agent can occur through the guidewire lumen in catheter systems as previously described having the guidewire lumen port located between a proximal balloon and one or more distal occlusion balloons.

In an embodiment in which the agent delivery lumen distal port (providing access to within the catheter shaft) is the only shaft port which is located between the proximal balloon and the first and second distal balloons, the treatment region between the inflated balloons is fully isolated, except for agent delivery port. Consequently, agent flowing from the agent delivery port does not leave the treatment region by flowing into or through additional accessible lumen(s) of the catheter. As a result, wash out is minimized or prevented and tissue uptake of the agent is enhanced. Additionally, by minimizing the number of lumens in the catheter shaft, the catheter has an improved low profile and maximizes the size of the inflation and agent delivery lumens, for improved catheter performance (e.g., shorter procedure time, enhanced track/distal small vessel access, and the like).

An alternative embodiment of the invention is directed to a porous balloon bifurcated catheter, generally comprising an elongated shaft having an inflation lumen, a guidewire lumen, a proximal shaft section, and a branched distal shaft section having a first branch and a second branch, and a first balloon portion on the first branch of the distal shaft section and a second balloon portion on the second branch of the distal shaft section, the balloon portions being porous and each having an interior in fluid communication with the inflation lumen, so that fluid agent from the inflation lumen inflates the porous balloon portions and exits the catheter through the porous balloon portions. The porous balloon portions are either the branched distal section of a single, forked balloon (e.g., a Y or V-shaped balloon), or two separate balloons mounted on a branched distal shaft section. In one embodiment, the catheter includes one or more occlusion balloons in addition to the porous balloon portions located distal or proximal thereto and configured to occlude the body lumen and thereby prevent or inhibit wash out of an agent delivered through the porous balloon(s).

In a method of using the porous balloon bifurcated catheter, the first porous balloon portion is inflated in a side branch vessel of the bifurcation and the second porous balloon portion is inflated in a main branch vessel of the bifurcation with the inflated porous balloon portions typically at the crux of the bifurcation of the body lumen, so that diseased tissue at the crux is exposed to a therapeutic agent exiting the catheter though the porous balloon portions, to thereby treat the diseased tissue.

In one embodiment, a balloon catheter of the invention is provided with a perfusion lumen, which extends, in part, in both the first and second branches of the distal shaft section, to provide for perfusion during the duration that the catheter balloon(s) are inflated in the body lumen. Thus, due to the branched nature of the catheter, the perfusion lumen has a branched distal section with first and second distal ports in fluid communication with a proximal port, in one embodiment.

Another embodiment of the invention is directed to a method of delivering an agent to a patient's body lumen, generally comprising delivering agent through an agent delivery lumen of an infusion catheter into a region of the body lumen isolated between two or more occlusion balloons of the catheter, such that fluid (e.g., blood, saline, and the like) in the isolated treatment region is purged by being displaced by the agent into a purging lumen of the catheter. Thus, with the infusion catheter balloons inflated, the increasing pressure caused by the infusion of the agent into the treatment region of the body lumen forces the trapped blood therein to flow back through the purging lumen and exit the catheter at a proximal purging port. The method therefore effectively increases the local drug concentration by preventing or minimizing dilution of the drug within the body lumen.

The purging method preferably involves the use of an infusion catheter having separate agent delivery and purging lumens extending from the proximal end of the catheter to a distal location between two or more occlusion balloons. As a result, the preferred method minimizes agent wash out within the body lumen, unlike methods as previously described in which the region is purged by infusing agent that displaces the blood distally past a partially inflated/noninflated distal occlusion balloon, and then isolated by fully inflating the distal occlusion balloon. In addition, in one embodiment the method includes purging any unpenetrated agent remaining in the treatment region prior to deflation of the balloons by infusing a displacing fluid (e.g., blood, saline, contrast, and the like) into the isolated treatment region of the body lumen, and thus effectively increases the drug delivery efficiency by minimizing systemic wash out.

The purging method provides for purging the isolated treatment region of the body lumen without aspirating the region. As a result, the method of the invention does not expose the treatment region to suction force, and therefore avoids the potential disadvantageous affects caused thereby. For example, the vacuum force of an aspirator can potentially flex or otherwise act upon the diseased vessel wall during aspiration, which is particularly to be avoided in certain disease states such as vulnerable plaque where destabilization of the plaque cap can result its rupture.

A variety of suitable agents can be delivered using the catheter(s) and method(s) of the invention, including therapeutic and diagnostic agents. The agents are typically intended for treatment and/or diagnosis of coronary, neurovascular, and/or other vascular disease, and may be useful as a primary treatment of the diseased vessel, or alternatively, as a secondary treatment in conjunction with other interventional therapies such as angioplasty or stent delivery. A variety of suitable therapeutic agents can be used including but not limited to thrombolytic drugs, anti-inflammatory drugs, anti-proliferative drugs, drugs restoring and/or preserving endothelial function, and the like. A variety of bioactive agents can be used including but not limited to peptides, proteins, oligonucleotides, cells, and the like. A variety of diagnostic agents that can be used according to the present invention. According to the present invention, agents described herein may be provided in a variety of suitable formulations and carriers including liposomes, polymerosomes, nanoparticles, microparticles, lipid/polymer micelles, complexes of agents with lipid and/or polymer, and the like.

A balloon catheter of the invention can be used as part of a variety of interventional procedures, including to deliver conventional or bifurcated stents (drug eluting or bare metal), pre or post dilatation, especially at the bifurcation, and the like.

The invention facilitates delivery of agents to complex regions of the body, providing for therapy thereof. A catheter of invention enhances tissue uptake and retention of drugs to enable treatment of vascular regions, including bifurcations, and preferably minimizes agent wash out. These and other advantages of the invention will become more apparent from the following detailed description of the invention and accompanying exemplary drawings.

### BRIEF DESCRIPTION OF THE DRAWINGS

FIG. 1 is an elevational view, partially in section, of a multi-balloon bifurcated catheter embodying features of the invention.

FIGS. 2-4 are transverse cross sectional views of the catheter of FIG. 1, taken along lines 2-2, 3-3, and 4-4, respectively.

FIG. 5 illustrates the catheter of FIG. 1 with the balloons inflated within a bifurcated body lumen.

FIGS. 5A and 5B are transverse cross-sectional views of the catheter of FIG. 5, taken along line 5A-5A and 5B-5B respectively.

FIG. 6 illustrates an alternative embodiment of a multi-balloon bifurcated catheter embodying features of the invention, having a purging lumen.

FIGS. 6A and 6B are a transverse cross sectional views of the catheter of FIG. 6, taken along lines 6A-6A and 6B-6B, respectively.

FIG. 7 is an elevational view, partially in section, of a porous balloon bifurcated catheter embodying features of the invention.

FIGS. 8-10 are transverse cross sectional views of the catheter of FIG. 7, taken along lines 8-8, 9-9, and 10-10, respectively.

### DETAILED DESCRIPTION OF THE PREFERRED EMBODIMENTS

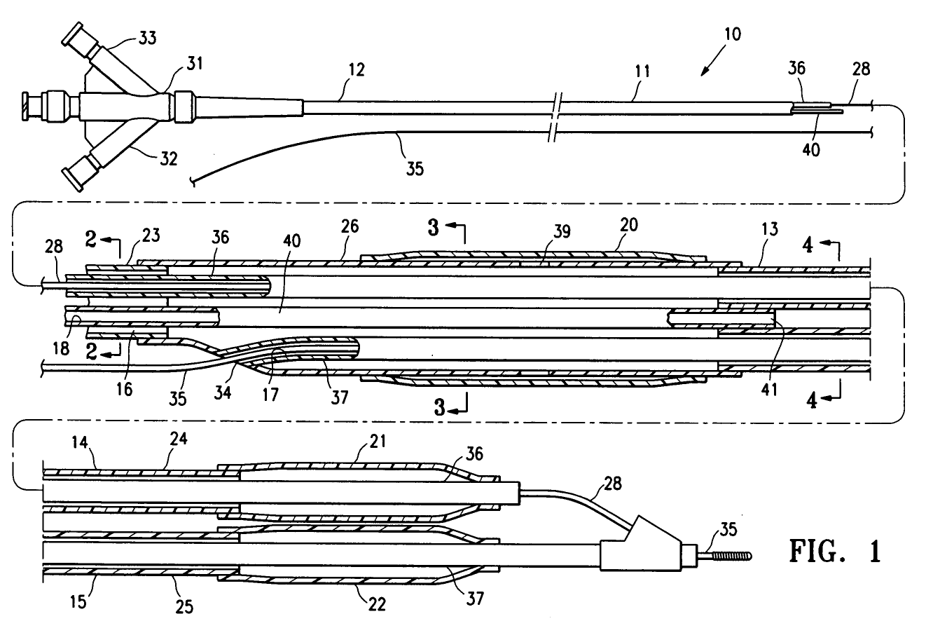

FIG. 1 illustrates an elevational view, partially in section, of a multi-balloon bifurcated catheter 10, embodying features of the invention, generally comprising an elongated catheter shaft 11 having a proximal end, a distal end, a proximal shaft section 12, a branched distal shaft section 13 with a first branch 14 and a second branch 15, an inflation lumen 16, a guidewire lumen 17, and an agent delivery lumen 18, and a proximal balloon 20 on the proximal shaft section, a first distal balloon 21 on the first branch, and a second distal balloon 22 on the second branch. FIG. 1 illustrates the balloons in a noninflated configuration, although the space between the inner surface of the noninflated balloons and the underlying section of the shaft may be somewhat exaggerated in FIG. 1 for ease of illustration.

In the embodiment of FIG. 1, the proximal shaft section 12 comprises a proximal outer tubular member 23 with a proximal section of the inflation lumen 16 therein, and the first branch 14 of the bifurcated distal shaft section 13 is formed in part by a first distal outer tubular member 24, and the second branch 15 is formed in part by a second distal outer tubular member 25. In the illustrated embodiment, the transition from the proximal shaft section to the distal shaft section comprises an intermediate outer tubular member 26 having a proximal end bonded to the distal end of the proximal tubular member 23, and a distal end bonded to the first and second distal outer tubular members 24, 25. However, a variety of suitable configurations can be used to transition from the proximal shaft section to the bifurcated distal shaft section, including alternative embodiments (not shown) in which the distal end of the proximal tubular member 23 is secured to the proximal end of the branched distal section, such that an intermediate section of the shaft is formed as an integral, one piece unit of a proximal tubular member.

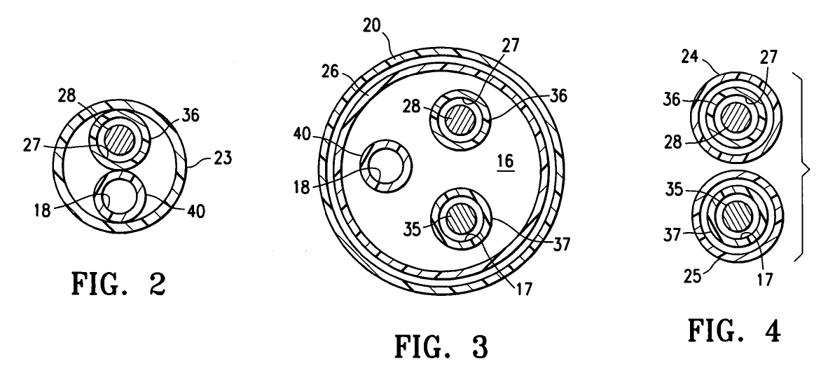

A joining wire lumen 27 (see FIG. 2), with a joining wire 28 slidably disposed therein, extends within the proximal section and the first branch 14 of the distal section to a distal end of the first branch. In the illustrated embodiment, the guidewire lumen 17, with guidewire 35 slidably disposed therein, is configured for rapid exchange and extends from a guidewire distal port at the distal end of the second branch 15 to a guidewire proximal port 34 in the proximal shaft section (through a side wall of the intermediate outer tubular member 26). A proximal adapter 31 is secured to the proximal end of the catheter shaft, which provides access to joining wire lumen 27, and which has a first arm 32 configured for connecting to a source of inflation fluid for inflating the balloons, and a second arm 33 configured for connecting to a source of agent.

In the illustrated embodiment, the first branch 14 comprises a first inner tubular member 36 defining the joining wire lumen 27 and first outer tubular member 24 defining, together with the outer surface of the inner tubular member 36 therein, the portion of the inflation lumen 16 in the annular space between the inner tubular member 36 and the outer tubular member 24 (see FIG. 4). The second branch 15 similarly comprises a second inner tubular member 37 defining the guidewire lumen 17 and outer tubular member 25 defining, together with the outer surface of the inner tubular member 37 therein, the portion of the inflation lumen 16 in the annular space therebetween.

The first and second branches 14, 15 of the branched distal shaft section 13 are preferably configured for releasably coupling together for introduction and advancement within a patient's body lumen. For example, in the embodiment of FIG. 1, the branches are in a coupled configuration with the distal end of the joining wire 28 slidably disposed in a coupler on the distal end of the second branch, and are uncoupled by sliding the joining wire 28 proximally out of the coupler. Typically, a connector (not shown) is provided on the proximal end of the adapter 31, facilitating proximally withdrawing the joining wire 28. Details regarding bifurcated catheter construction and use, and the coupling and uncoupling of the branches of a bifurcated catheter branched distal shaft section can be found in U.S. Pat. No. 6,017,324, incorporated by reference herein in its entirety. FIG. 1 illustrates the bifurcated catheter 10 in the coupled configuration.

The agent delivery lumen 18 is defined by an inner surface of a tubular member 40 extending within, and at least in part surrounded by, the inflation lumen 16 in the proximal shaft section 12. The agent delivery lumen 18 extends to a distal port 41 which is located adjacent to a proximal end of the branched distal shaft section 13, to thereby provide for delivery of an agent from the lumen 18, through port 41 and into a patient's body lumen (i.e., the distal agent port 41 opens to outside of the catheter). In the illustrated embodiment, the agent delivery lumen 18 extends to a single distal port (i.e., port 41). However, in alternative embodiments (not shown), the agent delivery lumen has multiple distal ports. For example, in one embodiment, the agent delivery lumen 18 is in fluid communication with multiple distal ports around the circumference and/or along a length (through the sidewall) of the distal end section of the tubular member 40, either in addition to the distal end port 41 in the distal end of tubular member 40, or instead of the distal end port 41 (such that the distal end port 41 of the tubular member 40 is optionally plugged). Although not illustrated, in one embodiment, the catheter 10 has one or more additional agent delivery lumens in addition to lumen 18, for example for the delivery of multiple component formulations.

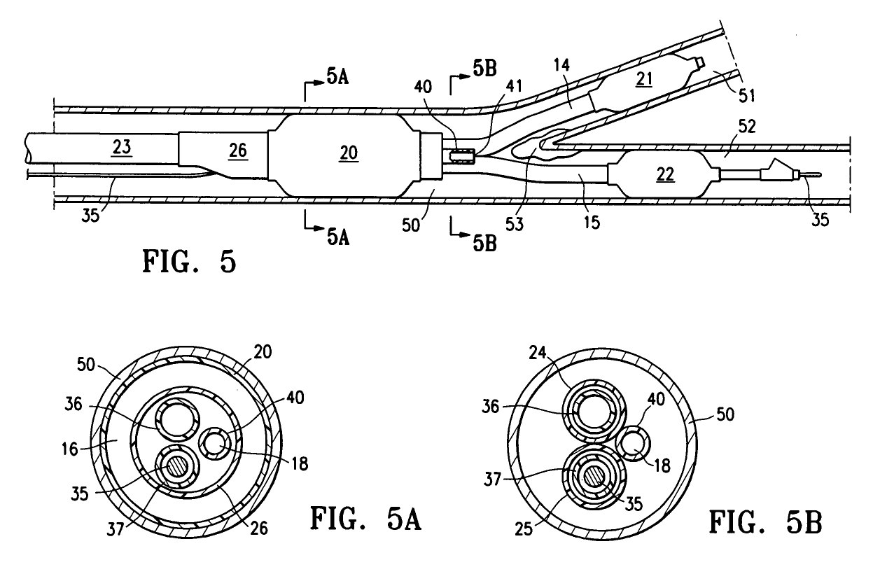

A distal portion of the tubular member 40 extends in side-by-side relation with a proximal end of the first and second distal outer tubular members 24, 25. In one embodiment, an outer surface of the tubular member 40 is in contact with and typically bonded to an outer surface of the tubular members 24, 25 (see FIG. 5B), although they may alternatively be radially spaced apart. The tubular member 40 and the first inner tubular member 36 are preferably eccentric, i.e., not coaxial, relative the inflation lumen in the proximal shaft section proximal to the branched distal section, see FIGS. 2 and 3, illustrating transverse cross sections of the catheter of FIG. 1, taken along lines 2-2 and 3-3, respectively. However, one or both of the tubular members 36 and 40 can be coaxial relative to the inflation lumen 16 in alternative embodiments (not shown).

In the illustrated embodiment, a proximal portion of the proximal shaft section, located proximally adjacent to the guidewire proximal port 34, has three lumens therein, namely, the inflation lumen 16, the agent delivery lumen 18, and the joining wire lumen 27. By limiting the number of lumens in the proximal portion of the proximal shaft section to, in one preferred embodiment, no more than three lumens, the catheter has a low profile with improved deliverability.

The first distal balloon 21 has a proximal end sealingly secured to a distal end of the first distal outer tubular member 24 and a distal end sealingly secured to a distal end of the first inner tubular member 36, and second distal balloon 22 has a proximal end sealingly secured to a distal end of the second distal outer tubular member 25 and a distal end sealingly secured to a distal end of the second inner tubular member 37, so that interiors of balloon 21 and balloon 22 are in fluid communication with the inflation lumen 16. In the illustrated embodiment, a port 39 in the side wall of intermediate tubular member 26 places the interior of proximal balloon 20 in fluid communication with the inflation lumen 16. Thus, the balloons 20, 21, 22 are in fluid communication with a common inflation lumen 16 for simultaneous inflation in the illustrated embodiment. However, in an alternative embodiment (not shown), the shaft has separate multiple inflation lumens providing for independent inflation of one or more of the balloons.

In a presently preferred embodiment, balloons 20, 21, 22 are solid walled occlusion balloons with fluid tight interiors, so that inflating the balloons occludes the body lumen. The occlusion balloons 20, 21, 22 can be formed of a variety of suitable materials commonly used to form catheter balloons, and are typically formed of elastomers such as Latex to provide high compliant balloons or non elastomers such as Nylon/Pebax to provide low compliant balloons which inflate into contact with the vessel wall to occlude the body lumen.

FIG. 5 illustrates the balloon catheter 10 of FIG. 1 with the balloons inflated in a patient's bifurcated body lumen -50. In FIG. 5, the first branch 14 of the catheter has been uncoupled from the second branch 15 and positioned within a side branch vessel 51 of the bifurcation. With the first distal balloon 21 inflated in the side branch vessel 51, and the second distal balloon 22 and the proximal balloon 20 inflated in the main branch vessel 52 of the bifurcation, the balloons occlude the body lumen 50 and isolate a region of the body lumen therebetween. Although not illustrated, in one embodiment, the catheter 10 includes one or more perfusion lumens extending from proximal of balloon 20 to distal of at least one and typically both distal balloons 21, 22, to prevent ischemia during the duration that the inflated balloons are occluding the body lumen.

The isolated region includes part of the side branch vessel 51 and main branch vessel 52, so that agent delivered through the agent delivery lumen 18 and out the distal port 41 to the body lumen 50 at the bifurcation is contained within both the main and side branch vessels 51, 52. As a result, the crux 53 of the bifurcation is exposed to the delivered agent. For example, in the illustrated embodiment, the crux 53 of the bifurcation is diseased, so that the balloons 20, 21, 22 are positioned proximal and distal to the crux 53 of the bifurcation such that they do not cover up all or part of the diseased tissue at the crux 53. Thus, the first and second distal balloons 21, 22 are preferably spaced distally a sufficient distance from the proximal end of the branched distal shaft section 13 such that the first and second distal balloons 21, 22 inflate without contacting one another and without contacting the crux 53 of the bifurcation. A method of the invention thereby exposes the crux 53 of the bifurcation to a delivered agent, without covering up or otherwise restricting the agent from accessing the tissue at the crux 53. After a sufficient treatment duration (e.g., typically about 0.1 to about 30 minutes, more typically about 1 to 10 minutes), the balloons are deflated and the catheter repositioned within or removed from the body lumen.

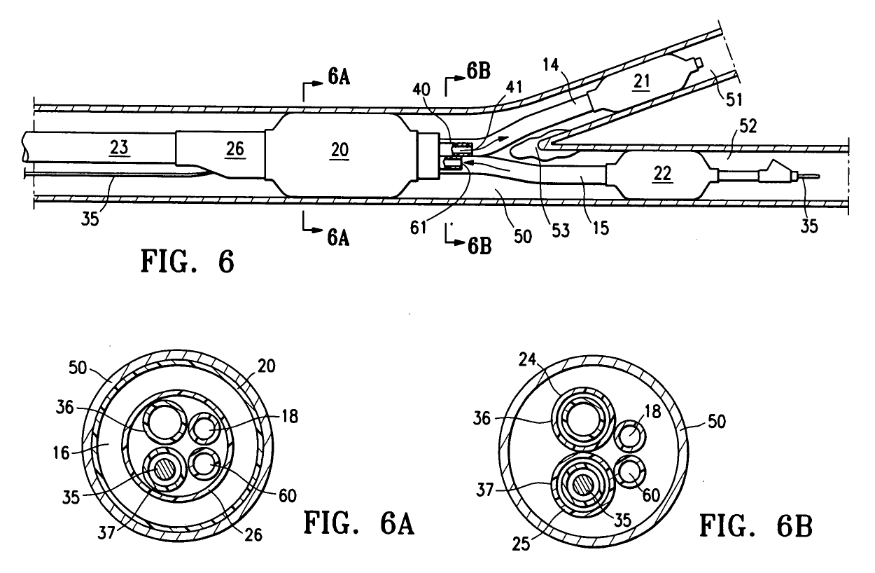

In the embodiment of FIG. 1, the distal port 41 of the agent delivery lumen 18 is the only shaft port (i.e., providing access from inside to outside of the catheter) located between the proximal balloon 20 and the first and second distal balloons 21, 22. FIG. 6 illustrates the distal end section of an alternative embodiment, in which a purging lumen 60 extends from the proximal end of the catheter to a distal port 61 located adjacent to the proximal end of the branched distal shaft section 13. The catheter of FIG. 6 is otherwise similar to the catheter of FIG. 1, with corresponding elements having the same reference numerals.

In a method of performing a medical procedure using a catheter having a distal port 61 of a purging lumen 60 adjacent to a distal port 41 of an agent delivery lumen 18, with the balloons inflated to isolate a treatment region therebetween, agent flows from the distal port 41 into the isolated treatment region. The purging lumen distal port 61 is preferably the only other shaft port located between the balloons, so that any fluid such as blood, saline, or contrast within the isolated treatment region is displaced by the agent and back-flows through the port 61 and out the catheter at the proximal end of the purging lumen 60. Although not illustrated, another proximal port is typically provided at the proximal adapter for collecting the fluid from the purging lumen 60. The fluid exiting from the proximal end of the purging lumen 60 will transition from being substantially fluid from the body lumen (e.g., blood, saline, and the like) to being substantially agent (i.e., substantially the solution/dispersion which contains the agent). At that point, the agent has displaced substantially all the fluid initially trapped between the catheter balloons, and the isolated region in now full of concentrated agent (i.e., agent which is not significantly diluted by the other fluid in the body lumen). In this way, the purging lumen allows for displacing of fluid from within the isolated treatment region without subjecting the region to the suctioning/vacuum force of an aspirator. After a sufficient treatment duration, the agent remaining within the isolated treatment region, which has not penetrated/adhered to the arterial wall tissue, can similarly be removed by flowing another fluid such as saline or contrast from the agent delivery lumen (or the purging lumen), so that the remaining agent is displaced and caused to back-flow through the purging lumen (or the agent delivery lumen). Flushing the remaining agent from the treatment region at the end of the treatment prevents the agent from flowing out of the treatment region after the balloons are deflated, and thus minimizes systemic wash out.

The distal ports 41, 61 are typically longitudinally staggered, with the purging lumen distal port 61 typically being longitudinally spaced from the agent delivery lumen distal port 41 by about 2 mm to about 5 cm. Although illustrated with the purging lumen distal port 61 as the more distal port, the agent delivery distal port 41 can alternatively be distal thereto in alternative embodiments (not shown). The staggered distal ports 41, 61 preferably prevent or minimize clogging of the lumens 40, 60 caused by biological or other clotting matter during agent infusion or flushing. For example, if both ports are very close to each other, one large clot or particulate could block both ports. Also a shear force could occur between in-flow and out-flow at the ports that could impact the flow rate of agent delivery or purging process.

Although discussed in terms of the embodiment of FIG. 6 having a proximal balloon proximal to first and second distal balloons on a branched distal section of the catheter shaft, it should be understood that a variety of suitable infusion catheters can be used for the purging method of the invention. For example, in one embodiment, an infusion catheter (not shown) has two balloons (i.e., a proximal and a distal balloon) which alone are sufficient for isolating a treatment region of the body lumen therebetween, for delivering an agent to a treatment region that is not bifurcated. Additionally, a catheter of the invention can be configured for performing procedures such as dilatation and stent delivery.

In a presently preferred embodiment, the purging method is performed using a catheter having multiple balloons in fluid communication with a common inflation lumen for simultaneous inflation of the balloons. Thus, the purging method of the invention allows for use of a lower profile, easier to manufacture catheter by avoiding the need for independent inflation of multiple balloons, resulting in improved, effective purging of an isolated treatment region. In contrast, prior purging methods require independent inflation of the proximal and distal balloons because blood in a partially isolated treatment region is displaced past a partially inflated/noninflated distal balloon until the treatment region appears under fluoroscopy to be filled with the infused agent (mixed with contrast), and the distal balloon is then fully inflated to isolate the treatment region.

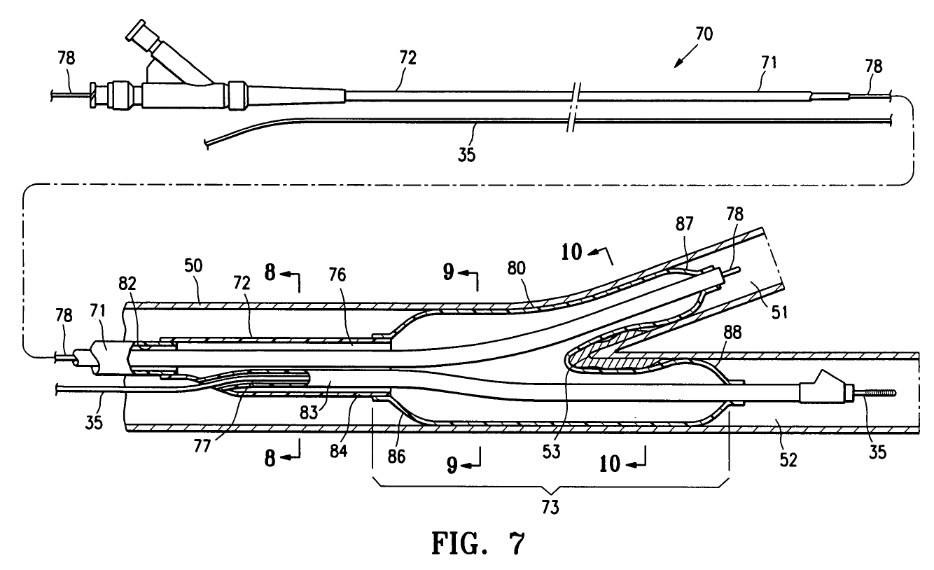

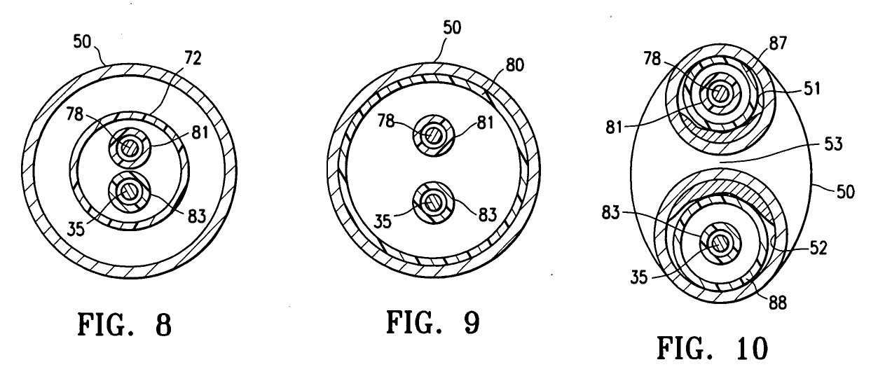

FIG. 7 illustrates an alternative embodiment of the invention, directed to a porous balloon bifurcated catheter 70, generally comprising an elongated catheter shaft 71 having a proximal shaft section 72, a bifurcated distal shaft section 73, an inflation lumen 76, and a guidewire lumen 77, and a forked balloon 80 with a branched distal section on the bifurcated distal shaft section. In the illustrated embodiment, the shaft has a first inner tubular member 81 defining a joining wire lumen 82 which is configured for wire 78, and a second inner tubular member 83 defining guidewire lumen 77, although a variety of suitable shaft designs can be used including a shaft having a bifurcated inner member (not shown). The first and second branches 87, 88 of the balloon 80 each have distal ends sealingly secured to the respective inner tubular members 81, 83 of the shaft, and come together to meet at a common proximal section 86 having a proximal end sealingly secured to the distal end of an outer tubular member 84 of the shaft, so that the interior of the balloon 80 is in fluid communication with the inflation lumen 76 of the shaft.

The balloon 80 is a porous balloon, so that fluid can be caused to flow across the balloon wall and into the body lumen 50. FIG. 7 illustrates the balloon 80 with the first branch 87 of the balloon distal section in a side branch vessel, and the second branch 88 of the balloon and the proximal section 86 of the balloon in a main branch of the body lumen. The balloon 80 is positioned so that the first and second branches 87, 88 of the balloon inflate into contact with the crux of the bifurcation of the body lumen. As a result, agent delivered from the porous balloon is delivered directly to the crux of the bifurcation and the, tissue of the vessel wall adjacent thereto at the bifurcation. In an alternative embodiment (not shown), a porous balloon bifurcated catheter of the invention has at least two separate porous balloons, namely a first porous balloon on the first branch of the distal shaft section and a second porous balloon on the second branch of the distal shaft section. The two balloons are preferably positioned on the catheter shaft such that proximal end sections of the two balloons inflate into contact with one another with the distal ends of the two balloons positioned in the side and main branch vessels of the body lumen. In the illustrated embodiment, agent in the balloon interior delivered from the inflation lumen flows through the porous balloon wall. However, a variety of suitable porous balloon configurations can be used including balloons having separate infusion lumens, and/or a porous outer layer allowing agent contained in a reservoir or otherwise delivered to the porous outer layer to flow through the porous outer layer. Although not illustrated, in one embodiment at least one of the first and second branch is provided with an occlusion balloon proximal or distal to the porous balloon portion, for occluding the body lumen during delivery of the agent from the porous balloon. For example, in one embodiment (not shown), a first distal occlusion balloon is provided on a distal extension of the first branch and a second distal occlusion balloon is provided on a distal extension of the second branch of the shaft, and are located distal to the porous balloon. Additionally, a porous balloon bifurcated catheter of the invention can be provided with one or more perfusion lumens configured to prevent ischemic conditions caused by inflation of the balloon(s) in the body lumen.

The dimensions of catheters 10/70 are determined largely by the size of the balloon and guidewire to be employed, the catheter type, and the size of the artery or other body lumen through which the catheter must pass or the size of a stent being delivered. By way of example, the proximal outer tubular member 23 typically has an outer diameter of about 0.025 to about 0.60 inch (0.064 to 0.15 cm), usually about 0.037 inch (0.094 cm), and a wall thickness of about 0.002 to about 0.008 inch (0.0051 to 0.02 cm), typically about 0.003 to 0.005 inch (0.0076 to 0.013 cm). The inner tubular member 36 typically has an inner diameter of about 0.01 to about 0.018 inch (0.025 to 0.046 cm), usually about 0.016 inch (0.04 cm), and a wall thickness of about 0.002 to about 0.004 inch (0.005 to 0.01 cm). The overall length of the catheter 10/70 may range from about 100 to about 150 cm, and is typically about 143 cm. Preferably, balloons 20, 21, 22 have a length about 0.8 cm to about 6 cm, and an inflated working (nominal) outer diameter of about 2 to about 5 mm.

The shaft tubular members can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives. Although the shaft is illustrated as having inner and outer tubular members, a variety of suitable shaft configurations may be used including a dual/multi-lumen extruded shaft having side-by-side lumens extruded therein. Similarly, although the embodiment illustrated in FIG. 1 is a rapid-exchange balloon catheter, the catheter of this invention may comprise a variety of intravascular catheters, such as an over-the-wire type balloon catheter having the guidewire lumen 17 extending the full length of the catheter 10.

While the present invention is described herein in terms of certain preferred embodiments, those skilled in the art will recognize that various modifications and improvements may be made to the invention without departing from the scope thereof. For example, although discussed primarily in terms of an embodiment in which a joining wire releasably couples the distal branches of the catheter together for delivery, a variety of suitable catheter configurations can be used including coupling the distal branches with releasable sheaths, and the like, as are conventionally known. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed is:

1. A multi-balloon bifurcated catheter, comprising:
a) an elongated shaft having an inflation lumen, a guidewire lumen, a proximal shaft section with a proximal section of the inflation lumen therein, a branched distal shaft section having a first branch with a first distal section of the inflation lumen and a second branch with a second distal section of the inflation lumen, the first and second distal sections of the inflation lumen being in fluid communication with the proximal section of the inflation lumen, and an agent delivery lumen extending in the proximal shaft section to a distal port which is located adjacent to a proximal end of the branched distal shaft section; and
b) a proximal balloon on the proximal shaft section located proximal to the distal port of the agent delivery lumen, a first distal balloon on the first branch, and a second distal balloon on the second branch, the proximal and first and second distal balloons each having an interior in fluid communication with the inflation lumen.

2. The catheter of claim 1 wherein the distal port of the agent delivery lumen is the only port of the shaft which is located between the proximal balloon and the first and second distal balloons.

3. The catheter of claim 1 wherein the agent delivery lumen has multiple distal ports located adjacent to a proximal end of the branched distal shaft section.

4. The catheter of claim 2 wherein the guidewire lumen extends from a distal port at a distal end of the second branch to a proximal port in the proximal shaft section located between the proximal balloon and the proximal end of the proximal shaft section.

5. The catheter of claim 1 wherein the catheter shaft includes a purging lumen extending from the proximal end of the shaft to a distal port located adjacent to the distal port of the agent delivery lumen, so that the distal ports of the purge lumen and agent delivery lumen are located between the proximal balloon and the first and second distal balloons.

6. The catheter of claim 1 wherein the proximal balloon and first and second distal balloons are solid walled occlusion balloons with fluid tight interiors.

7. The catheter of claim 1 wherein the first distal balloon has a proximal end sealingly secured to the first branch and the second distal balloon has a proximal end sealingly secured to the second branch, and the proximal balloon has a distal end spaced proximally apart from the first and second distal balloons.

8. The catheter of claim 1 wherein the first and second distal balloons have substantially equal inflatable lengths.

9. The catheter of claim 1 wherein the inflation lumen is eccentric to the agent delivery lumen throughout the length of the proximal shaft section.

10. The catheter of claim 1 wherein the agent delivery lumen is defined by an inner surface of a tubular member extending within and at least in part surrounded by the inflation lumen in the proximal shaft section.

11. The catheter of claim 10 wherein the first distal section of the inflation lumen is defined by a tubular member having a proximal end section with an outer surface in side-by-side relation to an outer surface of the agent delivery lumen tubular member at the distal port of the agent delivery lumen.

12. The catheter of claim 1 wherein the proximal balloon is proximally adjacent to the distal port of the drug delivery lumen and closer to the proximal end of the branched distal section than to the proximal end of the catheter shaft.

13. A method of performing a medical procedure, comprising:
a) introducing in a patient's body lumen a multi-balloon bifurcated catheter comprising
i) an elongated shaft having a proximal shaft section, a branched distal shaft section with a first branch and a second branch, and an agent delivery lumen extending in the proximal shaft section to a distal port which opens to outside of the catheter and which is located adjacent to a proximal end of the branched distal shaft section; and
ii) a proximal balloon on the proximal shaft section located proximal to the distal port of the agent delivery lumen, a first distal balloon on the first branch, and a second distal balloon on the second branch, the proximal and first and second distal balloons each having an interior in fluid communication with an inflation lumen;
b) advancing the catheter within the body lumen with the first and second branches releasably joined together to a bifurcation of the body lumen, and unjoining the branches, and positioning the first distal balloon in a side branch of the bifurcation and the second distal balloon in a main branch of the bifurcation;
c) inflating the proximal and first and second distal balloons; and
d) delivering an agent through the agent delivery lumen and out the distal port of the agent delivery lumen to the body lumen at the bifurcation, so that the agent is within the main and side branches of the bifurcation and between the inflated proximal and first and second distal balloons.

14. The method of claim 13 wherein the proximal balloon and first and second distal balloons are in fluid communication with a single inflation lumen in the proximal shaft section to thereby inflate simultaneously.

15. The method of claim 13 wherein the proximal and first and second distal balloons are solid walled occlusion balloons with fluid tight interiors, so that inflating the balloons occludes the body lumen.

16. The method of claim 13 wherein the first distal balloon has a proximal end sealingly secured to the first branch and the second distal balloon has a proximal end sealingly secured to the second branch at locations spaced distally a sufficient distance from the proximal end of the branched distal shaft section such that the first and second distal balloons are inflated without contacting oneanother, and without contacting an crux of the bifurcation of the body lumen.

17. The method of claim 13 wherein the distal port of the agent delivery lumen is the only shaft port opening to outside of the catheter which is located between the proximal and first and second distal balloons, so that agent delivered to the body lumen does not reenter the catheter shaft during infusion of the agent.

18. The method of claim 13 wherein the catheter shaft includes a purging lumen extending in the proximal shaft section to a distal port which is located adjacent to a proximal end of the branched distal shaft section, and wherein d) includes displacing fluid within the body lumen with agent by flowing said agent until fluid exiting from a proximal end of the purging lumen of the catheter transitions from being substantially fluid from the body lumen to substantially the agent.

19. A method of performing a medical procedure, comprising:
a) positioning within a patient's body lumen a distal section of a catheter shaft having at least a first proximal balloon and a second distal balloon, the shaft having an agent delivery lumen extending to a distal agent port between the proximal and distal balloons, and a purging lumen extending to a distal purging port between the proximal and distal balloons;
b) inflating the balloons to occlude the body lumen and isolate a treatment region between the balloons; and
c) delivering an agent through the agent delivery lumen such that the agent displaces fluid within the treatment region, by flowing said agent at least until displaced fluid back-flowing in the purging lumen and exiting from a proximal end of the purging lumen of the catheter transitions from being substantially fluid from the body lumen to substantially agent from the catheter.

20. The method of claim 19 including after c), flushing the treatment region, after delivery of the agent is stopped and before the balloons are deflated, by delivering fluid through the agent delivery lumen such that the fluid displaces excess agent remaining in the treatment region.

21. The method of claim 19 wherein the distal ports of the agent delivery lumen and the purging lumen are longitudinally staggered relative to one another, and c) includes delivering agent through the agent delivery lumen after the displaced fluid transitions from being substantially fluid from the body lumen to substantially agent.

22. A porous balloon bifurcated catheter, comprising:
a) an elongated shaft having an inflation lumen, a guidewire lumen, a proximal shaft section, a branched distal shaft section having a first branch and a second branch; and
b) a first balloon portion on the first branch of the distal shaft section and a second balloon portion on the second branch of the distal shaft section, the balloon portions being porous and each having an interior in fluid communication with the inflation lumen, so that fluid agent from the inflation lumen inflates the porous balloon portions and exits the catheter through the porous balloon portions.

23. The catheter of claim 22 wherein the catheter has a forked balloon with a branched distal section forming the first and second porous balloon portions, and with a proximal end sealingly secured to the catheter shaft.

24. The catheter of claim 22 wherein the catheter has a first balloon on the first branch of the distal shaft section forming the first porous balloon portion, and a second balloon on the second branch of the distal shaft section forming the second porous balloon portion and separate from the first balloon.

25. The catheter of claim 24 wherein the first and second balloon have proximal ends at a proximal end of the branched distal shaft section.

26. The catheter of claim 22 including a first occlusion balloon on the first branch of the distal shaft section located distal or proximal to the first porous balloon portion, and a second occlusion balloon on the second branch of the distal shaft section located distal or proximal to the second porous balloon portion.

27. The catheter of claim 22 including a proximal porous balloon on the proximal shaft section.
